(19) United States
(12) Patent Application Publication
(10) Pub. No.: US 2015/0223967 A1
(43) Pub. Date: Aug. 13, 2015

(54) DISPOSABLE DEVICE TO URINATE

(71) Applicant: Rafael ROJALS FORT, Tarragona (ES)

(72) Inventor: Rafael ROJALS FORT, Tarragona (ES)

(21) Appl. No.: 14/507,954

(22) Filed: Oct. 7, 2014

(30) Foreign Application Priority Data
Feb. 13, 2014 (ES) 201430200

(51) Int. Cl. A61F 5/451; A61F 5/453; A61F 5/455

(52) U.S. Cl. CPC A61F 5/451; A61F 5/4556; A61F 5/453; A61F 5/4404

(57) ABSTRACT

A disposable device for facilitating the action of urinating. The disposable device includes a tubular body of conical configuration, and is made from a laminar piece (2) and which has a wide mouth (3) at one of its ends recipient of the urine, and an evacuation or drainage opening (4) at the opposite end, and having between both ends a distance of approximately 10 to 12 cm. The wide mouth (3) is equipped with an elongated end (5) which can be extended or retracted and the extension or retraction thereof enables the device to adapt well to the female pelvic area or the male anatomy.

### OBJECT OF THE INVENTION

The invention, as stated in the heading of the present specification, relates to a disposable device for urinating which provides, to the function to which it is intended, advantages and characteristics of novelty that will be described in detail below and which involve an improvement compared with that currently known in the state of the art.

More in particular, the object of the invention centres on an device which has the purpose of facilitating the action of urinating, both in men and women, in men making it possible to extend the length of the penis with the aim of facilitating the action of urinating for those people who, due to various circumstances, cannot do so normally, and in women so that it can be done standing up by those who, due to physical problems, for comfort or due to other causes, cannot or do not want to do it sitting down, for which purpose it has the special characteristics of having a structural configuration especially designed for its adaptation to the female and male anatomy, of being designed to be of single use, of being manufactured in disposable material, preferably recyclable, such as cardboard or biodegradable plastic, and of having a size and shape which allow an easy and discrete transport.

### FIELD OF APPLICATION OF THE INVENTION

The field of application of the present invention centres on the sector of the industry devoted to the manufacturing of products for intimate hygiene and parapharmacy products.

### BACKGROUND OF THE INVENTION

As reference to the current state of the art, it should be mentioned that although in the market there are devices and devices intended to facilitate the action of urinating, at least by the applicant, the existence is unknown of any that has technical, structural and constitutional characteristics similar to those claimed herein, since they are normally designed for exclusively male use or are devised for exclusively female use.

In said sense, it should be mentioned that there is knowledge of the existence in the market of various types of devices which facilitate the urination of women so that they can do this standing up, as well as the existence of other specific devices for men, generally devised for bedridden persons or similar uses, since they usually consist of flamboyant contraptions of rubber or similar; however, none similar to that disclosed in the invention has been found nor with a purpose of facilitating the normal urination of men when he has difficulties to do so and, at the same time, serve so that women can urinate standing up.

As is known, there are multiple circumstances which prevent men from correctly urinating and in conventional form, i.e. standing up in front of the toilet or urinal. This may be due to different causes, either due to problems of length of the penis, or due to problems of mobility, especially in the elderly, or for other causes.

One of the objectives of the present invention is, therefore, to provide the market with a practical disposable device which, designed to barely occupy space and which can be discretely carried, is easy to use in any circumstance and place, avoiding uncomfortable and embarrassing situations for the users.

In parallel, a second objective of the present invention is to provide the same solution for women when they cannot or do not want to sit down on the toilet for urinating.

Finally, a last objective of the invention is to provide the market with an device of mixed use which allows the manufacturer to cover both men and women with the same product as target or market group.

### DESCRIPTION OF THE INVENTION

Thus, the disposable device for urinating that the present invention proposes is configured as a significant novelty within its field of application, since in accordance with its implementation and specific form it satisfactorily achieves the objectives indicated above, the characterizing details making it possible and distinguishing it from what is already known, conveniently included in the final claims which accompany the present specification thereof.

Specifically, what the invention claims is an device which is configured as a tubular body of conical structural configuration, made from a laminar piece of disposable material, such as of cardboard or biodegradable plastic, which has, at one of its ends, a wide mouth the dimensions of which are suitable to receive a penis and, at the same time, adapt to the pelvic area or genital area of a women where the external end of their urethra is found, and at the opposite end of said conical tubular body, which is considerably narrower, it has an evacuation or drain opening, having between both ends a distance of approximately 10 to 12 cm, in any case sufficient so that when urinating, the urine is evacuated forward a certain distance from the body, and both male and female users can urinate standing up.

It is important to indicate that, according to the essential characteristics of the invention, the wide mouth of the described conical tubular body which forms the claimed device, has a contour elongated by one of its ends to remain in concordance with the anatomy of the external genital area of a woman, so that it allows its adaptation adjusted thereto, also having provided folding lines which make it possible to modify the shape of said contour of the wide mouth so that, via a simple folding, it adapts to men's anatomy facilitating the use of the device for them.

Likewise, it should also be highlighted that the described tool, in addition to being made in a disposable material such as cardboard or plastic, since in any case it is a laminar piece structured in the form of a cone, is also advantageously foldable, so that its storage and transport is performed in said folded position, preferably being distributed and/or sold in packs of several units, allowing the user to be able to carry them at the same time in any pocket, bag, etc.

The described disposable device for urinating thus represents an innovative structure of structure and constitutional characteristics unknown until now for the purpose for which it is intended, reasons which, together with its practical use, give it sufficient grounds to obtain the privilege of exclusiveness applied for.

### DESCRIPTION OF THE DRAWINGS

To complement the description being made and in order to aid towards a better understanding of the characteristics of the invention, a set of drawings is attached as an integral part of said specification wherein, with illustrative and non-limiting character, the following has been represented:

FIG. 1.—Shows a perspective view of an example of embodiment of the disposable device for urinating, object of the invention, observing therein its general configuration, in said figure being represented in position of use for women, i.e. with the elongated end of the wide mouth unfolded.

FIG. 2.—Shows a perspective view of the female disposable device for urinating, according to the invention, in this case represented in the use position for men, i.e. with the elongated end of the wide mouth retracted.

FIG. 3.—Shows a diagrammatic view of a female silhouette with the disposable device for urinating represented in use position, observing in this case its adaptation to the female anatomy.

### PREFERRED EMBODIMENT OF THE INVENTION

In light of said figures, and in accordance with the numbering adopted, it is possible to observe in them an example of the disposable device for urinating claimed, which comprises the parts and elements which are indicated and described in detail below.

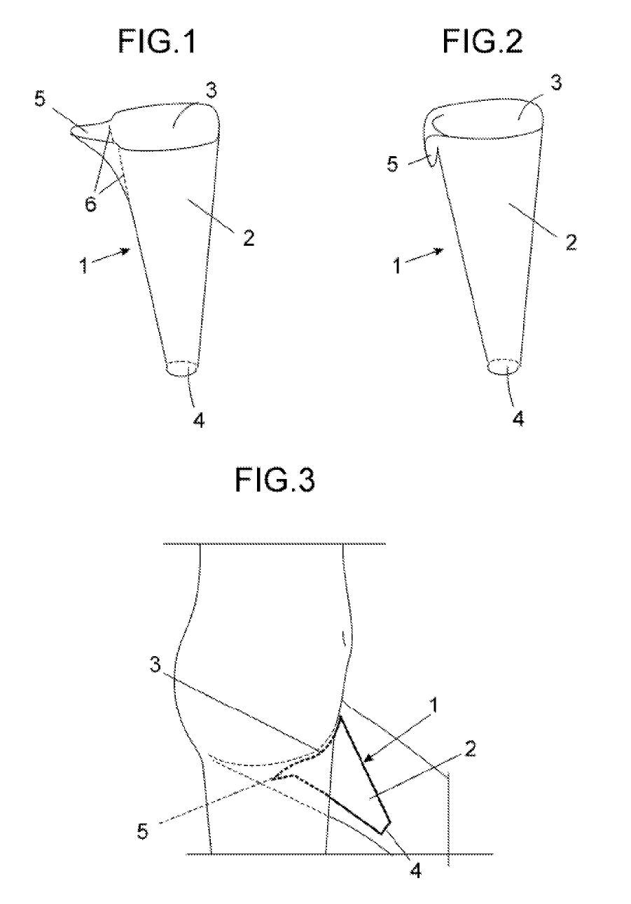

Thus, as observed in FIG. 1, the device (1) in question includes a tubular body of conical configuration, made from a curved laminar piece (2), of disposable material, preferably recyclable, such as cardboard or biodegradable plastic, with a wide mouth (3) at one of its ends recipient of the urine, and an evacuation or drainage opening (4) of said urine, at the opposite end, having between both ends a distance of approximately 10 to 12 cm, and where said wide mouth (3) has the special characteristic of being equipped with an elongated end (5) which can be extended or retracted and the extension or retraction whereof enables the device to adapt well to the female pelvic area or the male anatomy.

Preferably, to facilitate the extension or retraction of said elongated end (5), depending on whether the device is going to be used by a female or male user, the existence of folding lines (6) has been provided in the laminar body (2) which conveniently guide its position.

Finally, it should be highlighted that, as it is a laminar piece (2), it can be folded, making it adopt a completely flat configuration which minimizes its volume and facilitates its transport and storage.

Having sufficiently described the nature of the present invention, as well as the manner of putting it into practice, it is not considered necessary to give further explanation in order for any person skilled in the art to understand its scope and the advantages derived therefrom, stating that, within is essential nature, it can be put into practice in other forms of embodiment that differ in details from those indicated by way of example, and whereto protection claimed will equally cover, provided that its main principle is not altered, changed or modified.

1-5. (canceled)

6. A disposable device for urinating comprising:
a body having a conical configuration, the body is made from a laminar piece and includes:
a first end having a wide mouth adapted to receive urine, a second end including a drainage opening, an elongated end located on the wide mouth, the elongated end extends or retracts to adapt to either a female pelvic area or a male anatomy;
between the first end and the second end there is a distance of approximately 10 to 12 cm.

7. The disposable device according to claim 6, wherein to elongated end includes pre-existing folding lines to facilitate the extension or retraction of said elongated end of the wide mouth.

8. The disposable device according to claim 6, wherein the laminar piece is made of a recyclable material.

9. The disposable device according to claim 8, wherein the laminar piece is made of a cardboard.

10. The disposable device according to claim 8, wherein the laminar piece is made of a biodegradable plastic.
